(19) United States
(12) Patent Application Publication
(10) Pub. No.: US 2015/0136998 A1
(43) Pub. Date: May 21, 2015

(54) STERILIZER FOR WATER, WITH PUMP, PARTICULARLY FOR AQUARIUMS

(71) Applicants: Marco Pierobon, Padova (IT); Giacomo Guoli, Milano (IT); Fabio Bellia, Trebaseleghe (IT)

(72) Inventors: Marco Pierobon, Padova (IT); Giacomo Guoli, Milano (IT); Fabio Bellia, Trebaseleghe (IT)

(73) Assignee: NEWA-TECNO INDUSTRIA S.R.L., Loreggia (IT)

(21) Appl. No.: 14/400,895

(22) PCT Filed: May 14, 2012
(86) PCT No.: PCT/IB2012/052397

§ 371 Date: Nov. 13, 2014

(51) Int. Cl. A61L 2/10; C02F 1/32

(52) U.S. Cl. CPC A61L 2/10; C02F 1/325; C02F 2201/328
USPC 250/435

(57) ABSTRACT

A sterilizer for water, with pump, particularly for aquariums, comprising
at least one element for irradiation of ultraviolet rays,
and at least one duct portion for the passage of the water to be sterilized, which faces the irradiation element, the duct portion being permeable to ultraviolet radiation irradiated during use by the irradiation element,
a pump for supplying water to the duct portion,
a structure for hanging from a rim of a wall of a tank, which has a first supporting portion for the intake of the pump and a second supporting portion for the irradiation element and for the duct portion that faces it.

The present invention relates to a sterilizer for water, with pump, particularly for aquariums.

Nowadays, in the field of the production and sale of accessories for aquariums, ultraviolet sterilizers are known.

These comprise an ultraviolet lamp accommodated in a transparent jacket immersed in a sterilization tray through which the water to be sterilized flows, which has to be extracted from the tank of the aquarium and then returned to the tank.

In order to extract the water to be supplied to the sterilizer, currently an immersion pump is used, which is arranged inside the tank.

This pump is provided with means of fixing to the inside of the aquarium, such as for example a sucker adapted to enable the association thereof with the inner wall of the tank, under the surface of the water.

The sterilizer is fixed externally to the aquarium in a position sheltered from view or in any case such as to not engender a negative visual impact for the overall appearance of the aquarium.

The connection of the pump to the sterilizer is achieved by way of tubes which the installer has to connect to the delivery of the pump and to the inlet of the sterilizer.

Moreover, a return duct is provided, which is connected to the outlet of the sterilizer, and which is fixed to the aquarium so that its free end discharges back into the tank.

A drawback of such conventional structure consists in that its installation is usually carried out by a person who is not an expert or who is not skilled in mounting such devices.

In fact, nowadays the sterilizer, pump and tubes are generally available on the market separately and not pre-assembled, in order to make it possible for the length of the tubes to be adapted to the contingent conditions of installation.

This means that the installer, in order to enable an effective operation of the system installed, is required to have a sufficient manual ability to correctly make impermeable connections between the tubes and the pump and sterilizer, as well as the ability to connect these correctly to the aquarium.

Otherwise, an incorrect connection of such components can cause an incorrect operation thereof and water leaks which can be disastrous in terms of the damage that can result if the aquarium is placed for example in an environment with wood floors, or other floors capable of allowing infiltration to the floors below.

The aim of the present invention is to provide a sterilizer for water, with pump, particularly for aquariums, using ultraviolet irradiation, which is easier to install than the ultraviolet sterilizers known today.

Within this aim, an object of the invention is to provide a sterilizer for water, with pump, particularly for aquariums, which is more compact than the sterilizers known today.

Another object of the invention is to provide a sterilizer that does not require adaptation operations for its installation, such as for example the impermeable connection of tubes to other components.

Another object of the invention is to provide a sterilizer of the type that is ready for use, which does not require operations of adaptation or adjustment to the aquarium that is adapted to receive it as an accessory.

Another object of the invention is to provide a sterilizer that is structurally simple and easy to use, and which can be made at low cost.

This aim and these and other objects which will become better apparent hereinafter are achieved by a sterilizer for water, with pump, particularly for aquariums, comprising
at least one element for irradiation of ultraviolet rays,
and at least one duct portion for the passage of the water to be sterilized, which faces said at least one irradiation element, said at least one duct portion being permeable to ultraviolet radiation irradiated during use by said at least one irradiation element,
   characterized in that it comprises
a pump for supplying water to said at least one duct portion,
a structure for hanging from a rim of a wall of a tank, which has a first supporting portion for the intake of said pump and a second supporting portion for said at least one irradiation element and for said at least one duct portion that faces it.

Further characteristics and advantages of the invention will become better apparent from the description of a preferred, but not exclusive, embodiment of the sterilizer according to the invention, which is illustrated by way of non-limiting example in the accompanying drawings, wherein:

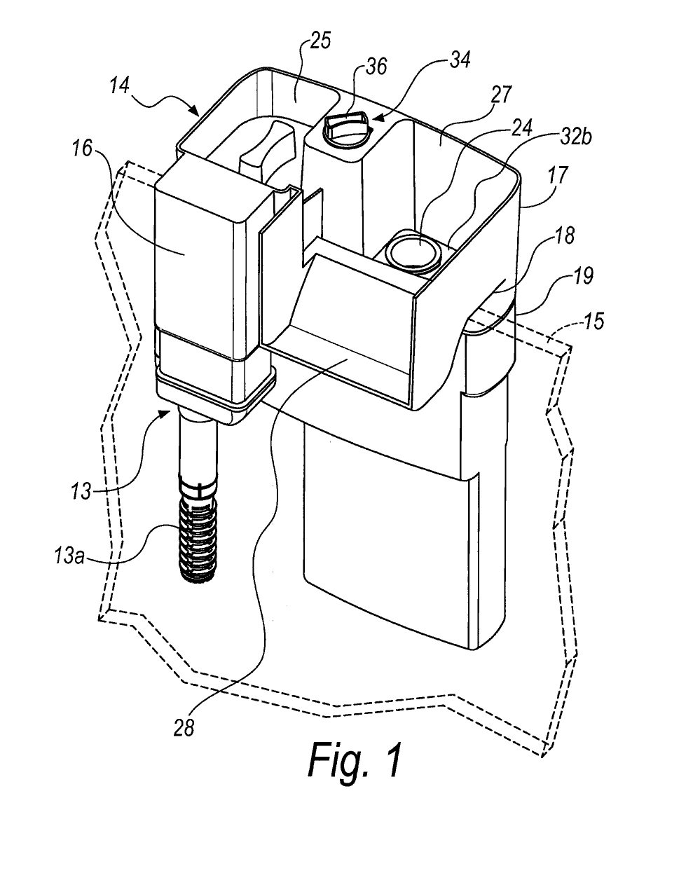

FIG. 1 is a perspective view of a sterilizer according to the invention;

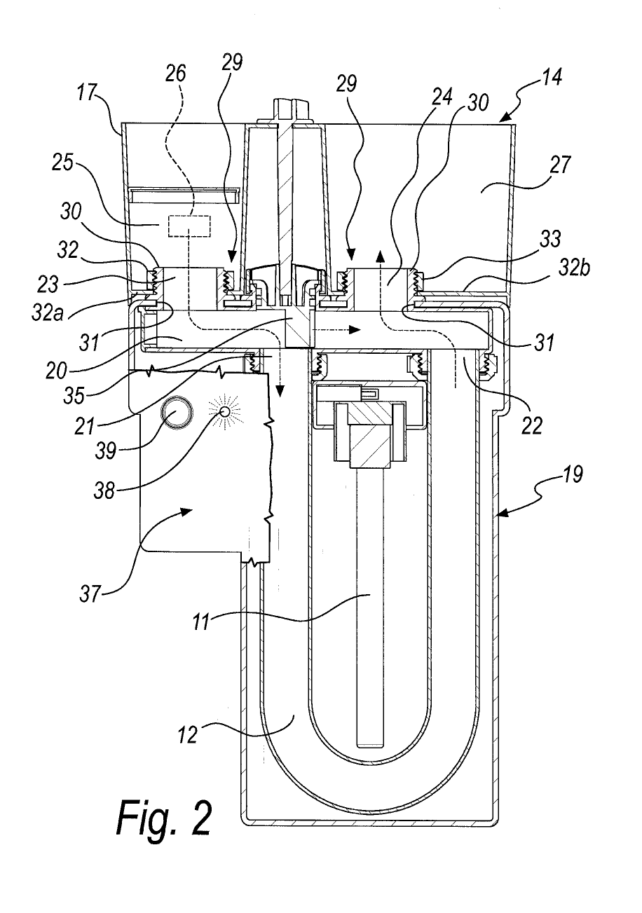

FIG. 2 is a cross-sectional view of a sterilizer according to the invention.

With reference to the figures, the reference numeral 10 generally designates a sterilizer for water, with pump, particularly for aquariums, comprising
an irradiation element 11 of ultraviolet rays, preferably of the UV-C type,
and a duct portion 12 for the passage of the water to be sterilized, which faces the irradiation element 11 and is permeable to ultraviolet radiation irradiated during use by the irradiation element 11.

In general, the duct portion is permeable to ultraviolet radiation at least in the part thereof that faces the irradiation element.

According to the invention, the sterilizer 10 has a particular peculiarity in that it further comprises
a pump 13 for supplying water to the duct portion 12,
a structure 14 for hanging from a rim 15 of a wall of an aquarium tank, which has a first supporting portion 16 for the intake 13a of the pump 13 and a second supporting portion 17 for the irradiation element 11 and for the duct portion 12 that faces it.

In alternative embodiments of the invention, in a substantially equivalent manner, in general there is at least one irradiation element and at least one duct portion which faces it and is permeable to the radiation emitted thereby in order to sterilize the water that transits it.

Advantageously, the hanging structure 14 has a central bridge-like portion 18 between the first portion 16 and the second portion 17, which is contoured for resting on the rim 15 of the tank so as to support
the intake 13 a of the pump 13 to draw from inside the tank, and support
the irradiation element 11 and the duct portion 12 outside the tank.

In the embodiment of the invention described and illustrated here by way of non-limiting example, the pump 13 is conveniently supported by the first portion 16, i.e. inside the tank during use.

However, in alternative embodiments of the invention, which are not shown in the accompanying figures or described below, the pump can be supported outside the tank during use.

Preferably, the sterilizer 10 comprises a shell 19, which accommodates the irradiation element 11 and the duct portion 12.

Inside the shell 19, there is conveniently moreover a manifold 20 onto which the inlet 21 and the outlet 22 of the duct portion 12 are open, and an access opening 23 and an outflow opening 24 for the water pumped by the pump 13.

The hanging structure 14 advantageously moreover has
a supply tray 25 for the manifold 20, onto which the delivery 26 of the pump 13 and the access opening 23 are open,
a discharge tray 27 for the manifold 20, onto which the outflow opening 24 is open.

The discharge tray 27 conveniently has a chute 28 for returning the water from the discharge tray 27 to the tank that it faces during use.

Conveniently, means 29 are provided for the reversible fixing of the shell 19 to the second supporting portion 17, which preferably comprise threaded collars 30, which protrude from the shell 19 and define the access opening 23 and the outflow opening 24, there being
holes 31 for the insertion of the threaded collars 30, which are defined on the bottoms 32a and 32b of the corresponding trays 25 and 27, and
rings 33 for locking the collars 30 to the bottoms 32a and 32b, which during use are screwed onto the collars 30 so as to abut against the bottoms 32a and 32b in order to retain the shell 19 thereon.

Advantageously, the sterilizer 10 moreover comprises a modulation device 34 for modulating the portion of water flow that is directed through the duct portion 12, in order to be sterilized.

Preferably, the modulation device 34 comprises a flow diverter 35, which is accommodated in the manifold 20 and rotatable therein in a manner that is modulable between
a first configuration, in which the flow diverter 35 substantially obstructs the direct passage through the manifold 20, from the access opening 23 to the outflow opening 24, while simultaneously allowing the passage from the access opening 23 to the outflow opening 24 substantially exclusively through the duct portion 12, as illustrated by way of non-limiting example in FIG. 2, and
a second configuration, in which the flow diverter 35 is arranged so as to allow easy passage for the water from the access opening 23 to the outflow opening 24 through the manifold 20,
and vice versa.

For example, the flow diverter 35 can have a semi-cylindrical structure with a diameter equal to the cross-sectional width of the manifold 20, and can be mounted so that it can axially rotate on the inlet 21 of the duct portion 12.

In this manner the rotation of the flow diverter 35 modulates the opening of the manifold 20, through which the water can pass, bypassing the duct portion 12.

Thus, in the first configuration substantially all of the water supplied by the pump 13 travels through the duct portion 12, and thus is exposed to the sterilizing radiation of the irradiation element 11.

In the second configuration, however, the water pumped by the pump will tend to pass through the manifold 20, bypassing the duct portion 12, and thus minimizing the effect of sterilization.

In intermediate configurations between the first and second configurations, the water supplied by the pump 13 is choked between
the duct portion 12, in which it is is sterilized, and
the manifold in which it is not sterilized,

thus obtaining a modulation of the sterilization effect depending on the choking of the flow that is induced in the duct portion 20 and is thus exposed to the radiation of the irradiation element 11.

The modulation device 34 advantageously comprises a knob 36 for actuating the flow diverter 35 which is on the outside of the shell 19, in order to enable the user to easily actuate the choking.

In an alternative embodiment of the invention, not shown in the accompanying figures, the irradiation element can be accommodated in a jacket that is permeable to ultraviolet radiation and is arranged in a channel which, together with the jacket, defines the duct portion.

Operation of a sterilizer according to the invention is the following.

In order to install it, it is sufficient to hang it with the central portion 18 resting on the rim 15 of the tank, so that the pump 13 draws from the water and the chute 28 is pointed toward it, the shell 19 protruding outside the tank.

Once the sterilizer 10 is connected to the mains electricity supply and actuated, the operator can modulate the sterilization effect by choking the water flow induced through the duct portion 20, by acting on the knob 36 that controls the flow diverter 35.

In particular, by way of example, in FIG. 2 a dashed line with arrows shows the route of the water flow choked through the duct portion 20.

Also in FIG. 2, an arrow and a dotted-and-dashed line show the route of the water flow through the manifold 20, when the flow diverter is rotated from the first configuration, in which it is shown in FIG. 2, to the second configuration.

The device according to the invention is provided with an “end of life” indicator of the irradiation element 11, which is accommodated in a compartment 37.

After a certain period of use, the irradiation element 11 decays and no longer emits sterilizing radiation, even if it remains switched on.

The electronic end-of-life device, not shown in detail, comprises a lapsed time meter that begins to count when the irradiation element 11 is inserted and switched on for the first time, and when the end of its lifetime is approaching, for example, an LED 38 flashes, and at its estimated end of effectiveness, the same LED 38 remains steadily lit, indicating the need to replace the irradiation element 11.

Once the spent irradiation element 11 is replaced with a new irradiation element 11, by way of a reset button 39 the lapsed time meter is set to zero, and restarts in order to track the lifetime of the new irradiation element 11.

In practice it has been found that the invention fully achieves the intended aim and objects, by providing a sterilizer for water, with pump, particularly for aquariums, by way of irradiation with ultraviolet rays, which is easier to install than the ultraviolet sterilizers known today, it being possible to simply hang it on the rim of the tank.

A sterilizer according to the invention is moreover more compact than the devices known today and does not require operations of adaptation for its installation, such as for example the impermeable connection of tubes to other components, which is required in the installation of devices known today.

Moreover, a sterilizer according to the invention is of the type that is ready to use, which does not require operations of adaptation or adjustment to the aquarium that is adapted to receive it as an accessory, it being possible to offer it for sale already assembled, or for assembly, separately supplying the pump, the hanging structure and the shell that accommodates the duct portion and the irradiation element that faces it, which can be easily assembled.

The sterilizer according to the invention is moreover structurally simple and easy to use, and can be produced at low cost.

The invention, thus conceived, is susceptible of numerous modifications and variations, all of which are within the scope of the appended claims. Moreover, all the details may be substituted by other, technically equivalent elements.

In practice the materials employed, provided they are compatible with the specific use, as well as the contingent dimensions and shapes, may be any according to requirements and to the state of the art.

1-10. (canceled)

11. A sterilizer for water, with pump, particularly for aquariums, comprising
at least one element for irradiation of ultraviolet rays,
and at least one duct portion for the passage of the water to be sterilized, which faces said at least one controlled irradiation element, said at least one duct portion being permeable to ultraviolet radiation irradiated during use by said at least one irradiation element, further comprising
a pump for supplying water to said at least one duct portion,
a structure for hanging from a rim of a wall of a tank, which has a first supporting portion for the intake of said pump and a second supporting portion for said at least one controlled irradiation element and for said at least one duct portion that faces it.

12. The sterilizer according to claim 10, wherein said hanging structure has a central bridge-like portion between said first portion and said second portion, which is contoured for resting on said rim of the tank so as to support said intake inside the tank and, outside said tank, support said at least one irradiation element and said at least one duct portion.

13. The sterilizer according to claim 10, further comprising a shell, which accommodates said at least one irradiation element and said at least one duct portion, inside said shell there being a manifold onto which the inlet and the outlet of said at least one duct portion are open, and an access opening and an outflow opening for the water pumped by said pump, said hanging structure having
a supply tray for said manifold, onto which the delivery of said pump and said access opening are open,
a discharge tray for said manifold, onto which said outflow opening is open, said discharge tray having a chute for returning water from said discharge tray to the tank that it faces during use.

14. The sterilizer according to claim 13, wherein it has means for the reversible fixing of said shell to said second portion.

15. The sterilizer according to claim 14, wherein said reversible fixing means comprise threaded collars, which protrude from said shell and define said access opening and said outflow opening, there being
holes for the insertion of said threaded collars, which are defined on the bottoms of said corresponding trays, and
rings for locking said collars to said bottoms, which during use are screwed onto said collars so as to abut against said bottoms in order to retain said shell thereon.

16. The sterilizer according to claim 10, further comprising a modulation device for modulating a portion of water flow that is directed through said at least one duct portion.

17. The sterilizer according to claim 16, wherein said modulation device comprises a flow diverter, which is accommodated in said manifold and rotatable therein in a manner that is modulable between
a first configuration, in which said flow diverter substantially obstructs the direct passage through said manifold, from said access opening to said outflow opening, while simultaneously allowing the passage from said access opening to said outflow opening through said at least one duct portion, and
a second configuration, in which said flow diverter is arranged so as to allow easy passage for the water from said access opening to said outflow opening through said manifold,
and vice versa.

18. The sterilizer according to claim 17, wherein said modulation device comprises a knob for the actuation of said flow diverter, which is exposed externally with respect to said shell.

19. The sterilizer according to claim 10, wherein said irradiation element is controlled by an electronic end-of-life device, which is provided with a lapsed time meter which is activated with the first switching-on of said irradiation element and indicates, by way of an LED (38), first the approach of the “end of life” and then the “end of life” of said irradiation element, with different indications.

20. The sterilizer according to claim 19, wherein said electronic end-of-life device is provided with a reset button, for setting the lapsed time meter to zero upon changing said irradiation element.
